(19) United States
(12) Patent Application Publication
(10) Pub. No.: US 2014/0157622 A1
(43) Pub. Date: Jun. 12, 2014

(54) Hand Dryer

(71) Applicant: Electrostar GmbH, Reichenbanch/Fils (DE)

(72) Inventor: Alexey Gorovoy, Krasnogorsky Rayon - Ilyinskoje (RU)

(21) Appl. No.: 13/939,219

(22) Filed: Jul. 11, 2013

(30) Foreign Application Priority Data
Jul. 11, 2012 (DE) 20 2012 006 801.6

(51) Int. Cl. A47K 10/00

(52) U.S. Cl. CPC A47K 10/00
USPC 34/572; 34/60

(57) ABSTRACT

A hand dryer has a housing and at least one exit opening communicating with an interior of the housing. At least one blower motor is arranged in the interior of the housing and conveys an air stream through the at least one exit opening to the exterior of the hand dryer. A supply device with at least one discharge opening is provided, wherein the supply device is configured to supply a disinfectant to the air stream through the at least one discharge opening.

### BACKGROUND OF THE INVENTION

The invention relates to a hand dryer comprising a housing in which at least one blower motor is arranged with which air is blown out to the exterior through at least one exit opening.

In hospitals, it is conventional that the physician washes his hands with soap and subsequently dries his hands with a towel or a hand dryer. Subsequently, a disinfectant from an arm-operated dispenser is dispensed onto the hands. The physician must wait until the hands are dry before he can put on sterile gloves. This procedure is very time-consuming and takes generally three to four minutes. When working in manipulation rooms, the physician must apply disinfectant onto his hands between procedures and must wait again until the hands are dry.

It is even more cumbersome that the washbasin, the hand dryer or the towel and the disinfectant dispenser are often arranged at different levels and are positioned at different locations. This is also a contributing factor in the time-consuming disinfection process.

It is an object of the present invention to design the hand dryer of the aforementioned kind in such a way that a hand disinfection can be done reliably within a short period of time.

### SUMMARY OF THE INVENTION

In accordance with the present invention, this is achieved in that a disinfectant can be supplied by means of at least one discharge opening to the air stream.

In the hand dryer according to the invention, the disinfectant is supplied to the air stream with which drying of the hands is performed. The disinfectant is dispensed through the at least one discharge opening of a supply device. The user therefore must not go to several different locations in order to dry his hands, disinfect them, and subsequently dry them again. By use of the hand dryer, the time for evaporation of the disinfectant is kept to a minimum so that the entire disinfecting sequence requires only little time. The disinfectant can be applied so as to be well distributed on the hands.

The disinfectant can be supplied to the air stream in the flow direction upstream or downstream of the exit opening. When the disinfectant is supplied upstream of the exit opening to the air stream, the disinfectant can be very finely dispersed so that the hands of the user are optimally wetted across the surface area. Moreover, the consumption of disinfectant can be kept at a minimum.

But even when the disinfectant is added downstream of the exit opening in the flow direction of the air stream, it is dispensed finely enough by the air stream so that a uniform wetting of the hands of the user is achieved while the consumption of the disinfectant is low.

In order to distribute the disinfectant optimally, the discharge opening is provided on a discharge nozzle which is present in a wall of an air guide of the hand dryer. The discharge nozzle can be designed such that the disinfectant reaches the air stream in a very finely dispersed state.

Advantageously, the discharge nozzle is provided in an area of the air guide in which the air is accelerated. Due to the high air speed, an optimal mixing of the air and the particles of the disinfectant exiting from the discharge nozzle is provided. This contributes to an optimal distribution of the particles in the air stream.

In an advantageous embodiment, a distributor e.g. in the form of a distributor plate is provided in the air guide and is positioned within the flow path of the air.

The distributor plate delimits in an advantageous way together with the wall of the air guide at least one flow space whose cross-section decreases in the flow direction of the air. Accordingly, in a constructively simple way, a high acceleration of the air stream within the air guide is achieved.

The flow space ends advantageously at a spacing upstream of the exit opening so that the air before exiting from the hand dryer is calmed; this leads to a uniform air flow.

The discharge nozzle for discharging the disinfectant is advantageously arranged in the area of this flow space so that the particles of disinfectant exiting from the discharge nozzle are reliably entrained by the accelerated air and dispersed in the air stream.

In a preferred embodiment, the hand dryer has several exit openings whose axes or exit flow directions are positioned angularly relative to each other. In this way, the air stream is divided into individual partial streams so that the hands of the user are reliably dried. Depending on the angular position of the exit openings, the air streams exiting therefrom can be guided such that they meet within a small or large area in which drying of the hands is performed.

Advantageously, the axes of the exit openings are positioned at acute angles relative to each other. In this way, for smaller dimensions of the hand dryer a large drying area can be generated.

In order to enable a simple and fast drying action of the hands, the air streams that are exiting from the exit openings meet in a drying area which is spaced from the exit openings.

The container for the disinfectant can be provided in the area adjacent to the housing or within the housing. When the container is arranged within the area adjacent to the housing, the container can be exchanged easily or refilled easily. When the container is located within the housing, the container is protected from damage and/or soiling.

In an advantageous embodiment, the hand dryer is provided with two independent contactless sensors with which the drying process and spraying of the disinfectant can be activated.

The disinfectant in an advantageous embodiment is sprayed on automatically after drying the hands.

In another advantageous embodiment, the hand dryer is designed such that it sprays the disinfectant with the beginning of the drying process onto the hands.

In accordance with a further preferred embodiment, the hand dryer can also be designed such that the user can activate spraying of the disinfectant as desired. The user has thus the possibility to apply the disinfectant before or after the drying process onto his hands or to eliminate the step of disinfection of the hands entirely.

A uniform spray application and a uniform drying action of the hands is achieved in an advantageous way by a simple constructive configuration of the hand dryer when the exit openings and the discharge nozzle for the disinfectant are positioned concentrically relative to each other.

When the hand dryer comprises a programmable microprocessor, different sequences for the drying process and the disinfection process can be set in a simple way. This programming function of the microprocessor can be designed such that the user himself can carry out programming in accordance with his own specifications.

The subject matter of the present application not only results from the individual claims but also from the features and specifications disclosed in the drawings and in the description. Even though these features and specifications may not be claimed, they are considered important to the invention inasmuch as they, individually or in combination, are new relative to the prior art.

Further features of the invention can be taken from the claims, the description and the drawings.

### BRIEF DESCRIPTION OF THE DRAWING

FIG. 1 is a schematic illustration in longitudinal section of a first embodiment of a hand dryer according to the invention.

FIG. 2 is a view in the direction of arrow II of FIG. 1.

FIG. 3 shows in an illustration in accordance with FIG. 1 a second embodiment of a hand dryer according to the invention.

### DESCRIPTION OF THE PREFERRED EMBODIMENTS

The hand dryer is designed such that it cannot only be used for drying but also for disinfecting the hands. The hand dryer is attached on a mounting plate 1, for example, that is fastened to a wall or the like, for example, by a screw connection. The hand dryer has a housing 2 in which a blower motor 3 is arranged with which, as is known in the art, an air stream is generated that exits at the bottom from the housing 2.

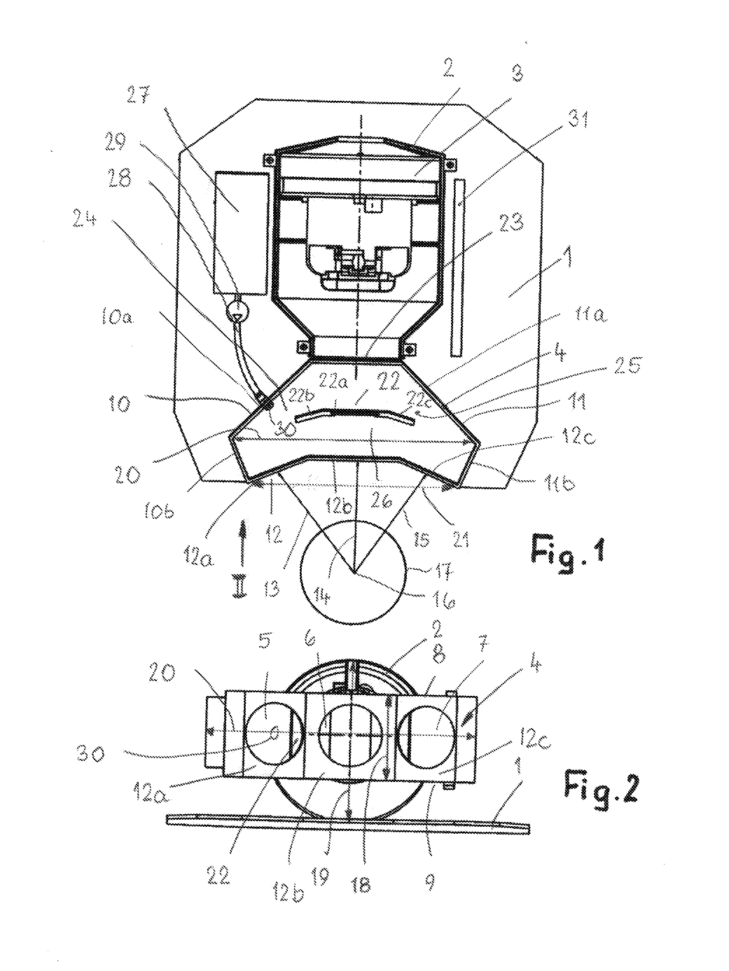

An air guide 4 is connected to the bottom end of the housing 2 and is provided with a rectangular cross-section (FIG. 2). The air guide 4 has three exit openings 5, 6, 7. They each have a circular cross-section and advantageously are of the same size.

The air guide 4 widens initially continuously away from the housing in the flow direction of the air. The air guide 4 has parallel longitudinal sidewalls 8, 9 that are connected to each other by narrow sidewalls 10, 11. The sidewalls 10, 11 are comprised of two wall sections 10a, 10b; 11a, 11b that are positioned at an obtuse angle relative to each other, respectively. The wall sections 10a, 11a adjoin the exit end of the housing 2 and diverge in the flow direction of the air. The wall sections 10a, 11a are longer than the wall sections 10b, 11b that converge relative to each other in the flow direction of the air. The wall sections 10b, 11b are connected to each other by a terminal wall 12 that connects also the longitudinal sidewalls 8, 9 of the air guide 4 with each other. The terminal wall 12 is comprised of three wall sections 12a, 12b, 12c that are positioned at an obtuse angle relative to each other. The wall sections 12a, 12c adjoin approximately at a right angle the wall sections 10b, 11b and diverge in the flow direction of the air. The central wall section 12b is positioned centrally and perpendicularly to the longitudinal axis of the housing 2.

Each wall section 12a to 12c is provided with one of the exit openings 5 to 7. Since the wall sections 12a to 12c are angularly positioned relative to each other, the air generated by the blower motor 3 exits in three air streams 13 to 15 that are positioned angularly relative to each other from the air guides 4. In FIG. 1, these three different air streams are illustrated as a straight line, respectively, for reasons of simplicity. The wall sections 12a to 12c and the exit openings 5 to 7 provided therein are oriented such that the air streams 13 to 15 meet each other in a drying location 16. In this area, the hands to be dried are located when using the hand dryer. The wall sections 12a to 12c are oriented such that the spacing of the drying location 16 from the exit openings 5 to 7 is only so large that a satisfactorily strong air stream will impinge on the hands to be dried so that an effective drying action is ensured. The hands to be dried are within the drying area that is schematically indicated by the circle 17 in FIG. 1 that is so large that the hands are completely or to a significant degree enclosed therein. The air streams 13 to 15 advantageously generate in the drying area 17 a swirl field that contributes to a fast drying action.

The air guide 4 has a width 18 that is smaller than the width 19 of the housing 2. The housing 2 has in the illustrated embodiment advantageously a circular cross-section so that the width 19 corresponds to the diameter of the housing 2. The housing 2 however can also have any other suitable cross-section.

The maximum length 20 of the air guide 4 is greater than the housing width 19. In the exit area, the length 21 of the air guide 4 is also greater than the housing width 19.

It should be noted that the described dimensional conditions are not mandatory but that the air guide 4 can also have different dimensions, depending on the field of use of the hand dryer. Also, the described configuration of the air guide 4 is advantageous but the air guide as a whole can also be of a different configuration. It is possible that the air guide 4 has at the exit end only a single exit opening, only two exit openings, or more than three exit openings. The air guide and the appropriate number of exit openings are to be provided always such that the hands located within the drying area 17 are dried quickly and reliably within a short period of time.

Within the air guide 4 there is a distributor plate 22 through which the air that is exiting from the exit end 23 of the housing 2 and flows into the air guide 4 is distributed such that from each one of the exit openings 5 to 7 the same air quantity exits, respectively. The distributor plate 22 extends between the two longitudinal sidewalls 8, 9 of the air guide 4 and is attached thereto in a suitable way. The distributor plate 22 is positioned centrally in the air guide 4 and has a central section 22a that extends parallel to the central wall section 12b and is located in the area above the central exit opening 6. The central plate section 22a is adjoined by the sections 22b and 22c at an obtuse angle, respectively, that diverge in the direction toward the terminal wall 12 of the air guide 4. The sections 22b, 22c are positioned at a spacing opposite the wall sections 10a, 11a and at an acute angle relative thereto. In this way, between the sections 22b, 22c and the wall sections 10a, 11a positioned at a spacing opposite thereto, a flow space 24, 25 is formed, respectively, which narrows in the flow direction of the air. Accordingly, the air in the flow spaces 24, 25 is accelerated. Advantageously, the exit speed is greater than approximately 35 m per second.

The distributor plate 22 ensures that the air stream that is exiting from the exit end 23 of the housing is distributed uniformly so that through the exit openings 5 to 7 approximately the same air quantities are exiting. The air that is flowing through the flow spaces 24, 25 flows into a common flow space 26 which is located in the area above the exit openings 5 to 7 and, in the flow direction, is delimited in downward direction by the terminal wall 12 and, in opposite direction thereto, partially by the distributor plate 22.

The air stream is heated, as is known in the art, for which purpose the hand dryer is provided with a hot air flow controller.

In the illustrated embodiment, the exit openings 5 to 7 are arranged in a line adjacent to each other (FIG. 2). However, it is also possible to arrange the exit openings such that their axes are positioned on a circle, for example. In this context, the axes are positioned angularly relative to each other such that the partial air streams meet within the drying area 17.

The number of exit openings depends also on their diameter. The smaller the opening diameter of the exit openings 5 to 7, the more exit openings are provided so that the required air quantity reaches the drying area 17.

The exit openings 5 to 7 are of a nozzle configuration and, apart from the disclosed shape, can also be designed as grid or aperture plates or as slits. The exit openings can also be provided in the form of a plurality of small bores and/or small slits in the terminal wall 12.

The arrangement of the exit openings 5 to 7 is selected advantageously such that the air streams will exit from the exit openings slightly at a slant away from the user.

A disinfectant is supplied to the air stream by means of a supply device; the disinfectant is contained in a reservoir 27 that is formed, for example, as a container that can be easily refilled or also exchanged when the disinfectant has been spent. In order to enable a simple exchange or refill, the reservoir 27 of the supply device is advantageously secured adjacent to the housing 2 on the mounting plate 1. In principle, the reservoir 27 can also be arranged within the housing 2. The term disinfectant is to be understood in the following as a solvent and the disinfecting agent contained therein.

To the reservoir 27 at least one line 28 of the supply device is connected by means of a valve 29 of the supply device. At the free end of the line 28 there is a discharge nozzle 30 which is attached on the wall section 10a of the air guide 4 and from which the disinfectant is discharged. The discharge nozzle 30 of the supply device is designed such that the exiting disinfectant reaches the partial air stream that flows through the flow space 24. Since the flow speed as a result of the cross-sectionally reduced section of the flow space 24 is high, the disinfectant upon exiting from the discharge nozzle 30 is entrained and dispersed finely in the air stream. Accordingly, the disinfectant exits finely dispersed through the exit openings 5 to 7 and is applied uniformly to the hands to be disinfected that are located in the drying area 17.

FIG. 1 shows schematically the electronic unit 31 with which the hand dryer is actuated.

In the illustrated embodiment, only one discharge nozzle 30 for the disinfectant is provided. It is also possible to provide the oppositely positioned wall section 11a in the same way with a further discharge nozzle so that the disinfectant can be supplied at two locations into the air stream. This additional discharge nozzle can be connected by a further conduit to the reservoir 27. In principle, it is however also possible to provide an additional reservoir with disinfectant for the additional discharge nozzle.

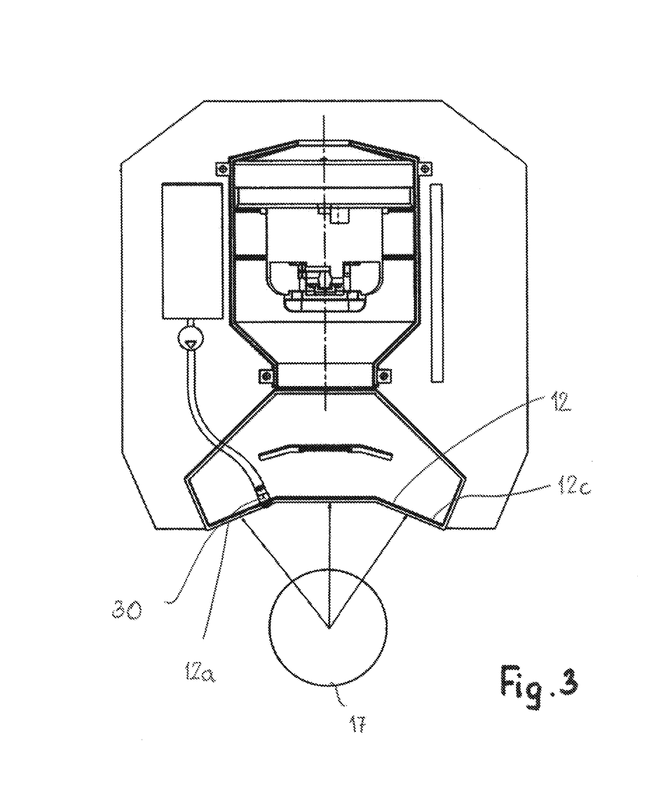

In the embodiment according to FIG. 3, the discharge nozzle 30 is attached to the wall section 12a and arranged such that the disinfectant is sprayed immediately onto the hands which are located within the drying areas 17. The discharge nozzle 30 in this case is attached in the area adjacent to the appropriate exit opening to the terminal wall 12. As in the preceding embodiment, a further discharge nozzle can also be provided in the wall section 12c so that the disinfectant is sprayed from two locations into the drying area 17.

With the described hand dryer it is not only possible to dry the hands after a washing step but also to disinfect and subsequently dry them. The electronic unit 31 is designed such that different procedures for use of the hand dryer are possible. The hand dryer is used, for example, in manipulation rooms and operating rooms in hospitals, in the field of veterinary medicine, and in places where a physician uses sterile gloves.

The hand dryer is advantageously designed such that it is switched on automatically when the hands are located within the drying area 17. Such an automatic actuation is known in connection with hand dryers and is therefore not explained in detail in this context. As soon as the hands are removed from the drying area 17, the hand dryer is automatically switched off.

The electronic unit 31 in a first embodiment can be designed such that first by means of the blower motor 3 the drying air is generated that exits from the exit openings 5 to 7 and impinges on the hands that are located in the drying area 17 so that they are dried. After a predetermined amount of time, the drying temperature is reduced and also the flowspeed of the drying air is lowered and then automatically the supply of disinfectant is switched on. The reservoir 27 has correlated therewith a metering device (not illustrated) with which the disinfectant is conveyed from the reservoir 27 under pressure into the conduit 28. For this purpose, the valve 29 is controlled so as to open for an appropriate amount of time. The disinfectant is sprayed by means of the discharge nozzle 30 into the air stream and the air stream finely disperses the disinfectant in a satisfactory way as a result of its appropriately high speed. The mixture of air and disinfectant passes through the exit openings 5 to 7 and reaches the hands located in the drying area 17. After a predetermined amount of time, the supply of the disinfectant is automatically switched off and only the drying air is continued to be supplied until the solvent has evaporated. Advantageously, the drying air is supplied until the hands are removed from the drying area 17 and the blower motor 3 is then automatically switched off. During the drying phase after the disinfection, it can be advantageous when the flow speed of the drying air is increased again.

In the embodiment according to FIG. 3, the disinfectant is not introduced into the air stream within the air guide 4 but immediately sprayed in the direction toward the drying area 17. The disinfectant is entrained by the air streams that are exiting from the exit openings 5 to 7. In other respects, the drying process is carried out in the same way as described above. In this embodiment, it is also possible to first spray on the disinfectant onto the hands and only then generate the drying air. The hand dryer in this case is designed such that the user first triggers the spraying action of the disinfectant. Then the blower motor 3 is switched on by time control or is switched on by the user.

The hand dryer can also be designed such that it performs only a disinfecting process. As soon as the hands have reached the drying area 17, the supply of disinfectant from the reservoir 27 is automatically turned on so that the disinfectant is sprayed into the air stream within the air guide (FIGS. 1 and 2) or directly sprayed onto the hands located within the drying area 17. Subsequently, hand drying is performed in that the supply of the disinfectant is switched off and only the drying air is conveyed through the exit openings 5 to 7 into the drying area 17. In this context, the solvent of the disinfectant is evaporated from the hands and the hands are thus dried. As soon as the disinfected hands have been pulled away from the drying area 17, the blower motor 3 is switched off.

The supply of disinfectant through the hand dryer has the advantage that the disinfectant is reaching the hands in a finely dispersed way. The drying air ensures a short drying action of the hands. Since the disinfectant is sprayed through the discharge nozzle 30, disinfectant is saved. Because of the spraying process, the hands to be disinfected are optimally uniformly sprayed with the disinfectant so that disinfection can be realized in a reliable way.

The hand dryer can be furnished with a timer, an air flow controller, or a hot air flow controller.

The drying process and the spraying action of the disinfectant can advantageously be activated by two independent contactless sensors. As soon as the hands reach the drying area 17, by means of these contactless sensors the blower motor 3 is switched on and the disinfectant is sprayed. Such a control of the hand dryer is possible in both described embodiments according to FIGS. 1-2 and according to FIG. 3.

It is moreover possible to design the hand dryer such that spraying of the disinfectant is activated when desired by the user. In this case, the user can determine himself whether the disinfectant is to be applied before or after the drying process or whether it is not at all applied to the hands.

Advantageously, the discharge nozzles 30 can be concentrically arranged so that the hands are uniformly subjected to the air stream and uniformly sprayed with the disinfectant during the drying process.

The hand dryer can be provided with a microprocessor with which different sequences for the drying process and the disinfection process can be programmed.

The specification incorporates by reference the entire disclosure of German priority document 20 2012 006 801.6 having a filing date of Jul. 11, 2012.

While specific embodiments of the invention have been shown and described in detail to illustrate the inventive principles, it will be understood that the invention may be embodied otherwise without departing from such principles.

What is claimed is:

1. A hand dryer comprising:
a housing;
at least one exit opening communicating with an interior of the housing;
at least one blower motor arranged in the interior of the housing and conveying an air stream through the at least one exit opening to the exterior of the hand dryer;
a supply device comprising at least one discharge opening, wherein the supply device is configured to supply a disinfectant to the air stream through the at least one discharge opening.

2. The hand dryer according to claim 1, wherein, in a flow direction of the air stream, the disinfectant is supplied to the air stream upstream of the at least one exit opening.

3. The hand dryer according to claim 1, wherein, in a flow direction of the air stream, the disinfectant is supplied to the air stream downstream of the at least one exit opening.

4. The hand dryer according to claim 1, further comprising an air guide with a wall, wherein the supply device has a discharge nozzle that is arranged in the wall of the air guide, wherein the at least one discharge opening is provided in the discharge nozzle.

5. The hand dryer according to claim 4, wherein the discharge nozzle is provided in an area of the air guide, wherein in said area the air is accelerated.

6. The hand dryer according to claim 4, further comprising a distributor plate arranged in the air guide in a flow path of the air stream.

7. The hand dryer according to claim 6, wherein the distributor plate and the wall of the air guide together delimit at least one flow space having a cross-section decreasing in a flow direction of the air stream.

8. The hand dryer according to claim 7, wherein the flow space is ending at a spacing upstream of the at least one exit opening.

9. The hand dryer according to claim 7, wherein the discharge nozzle is arranged in the area of the flow space.

10. The hand dryer according to claim 1, comprising several of the at least one exit opening, wherein said several exit openings each have an axis in an exit flow direction of the air and the axes are angularly positioned relative to each other.

11. The hand dryer according to claim 10, wherein the axes of said exit openings are positioned at an acute angle relative to each other.

12. The hand dryer according to claim 10, wherein partial air streams exiting from said several exit openings meet in a drying area of the hand dryer, the drying area positioned spaced apart from said several exit openings.

13. The hand dryer according to claim 1, further comprising a container for the disinfectant, the container arranged adjacent to the housing.

14. The hand dryer according to claim 1, further comprising a container for the disinfectant, the container arranged in the interior of the housing.

15. The hand dryer according to claim 1, further comprising two independent contactless sensors for activation of a drying process of the hand dryer and of a spraying action for the disinfectant.

16. The hand dryer according to claim 1, wherein the disinfectant is supplied automatically after drying of the hands of a user.

17. The hand dryer according to claim 1, wherein the disinfectant is sprayed onto the hands at the beginning of a drying process.

18. The hand dryer according to claim 1, wherein spraying of the disinfectant is activated by a user as desired.

19. The hand dryer according to claim 1, wherein the at least one exit opening and the discharge opening for the disinfectant are positioned concentrically to each other.

20. The hand dryer according to claim 1, further comprising a programmable microprocessor that is configured to enable programming of different sequences of drying and disinfection.
